(19) United States
(12) Patent Application Publication
(10) Pub. No.: US 2021/0319847 A1
(43) Pub. Date: Oct. 14, 2021

(54) PEPTIDE-BASED VACCINE GENERATION SYSTEM

(71) Applicant: NEC Laboratories America, Inc., Princeton, NJ (US)

(72) Inventors: Renqiang Min, Princeton, NJ (US); Wenchao Yu, Plainsboro, NJ (US); Hans Peter Graf, South Amboy, NJ (US); Igor Durdanovic, Lawrenceville, NJ (US)

(21) Appl. No.: 17/197,166

(22) Filed: Mar. 10, 2021

Related U.S. Application Data
(60) Provisional application No. 63/009,690, filed on Apr. 14, 2020.

(51) Int. Cl. G16B 15/30; G16B 40/00; G06N 3/08; G06N 3/04

(52) U.S. Cl. CPC G16B 15/30; G06N 3/0454; G06N 3/08; G16B 40/00

(57) ABSTRACT

A method is provided for peptide-based vaccine generation. The method receives a dataset of positive and negative binding peptide sequences. The method pre-trains a set of peptide binding property predictors on the dataset to generate training data. The method trains a Wasserstein Generative Adversarial Network (WGAN) only on the positive binding peptide sequences, in which a discriminator of the WGAN is updated to distinguish generated peptide sequences from sampled positive peptide sequences from the training data, and a generator of the WGAN is updated to fool the discriminator. The method trains the WGAN only on the positive binding peptide sequences while simultaneously updating the generator to minimize a kernel Maximum Mean Discrepancy (MMD) loss between the generated peptide sequences and the sampled peptide sequences and maximize prediction accuracies of a set of pre-trained peptide binding property predictors with parameters of the set of pre-trained peptide binding property predictors being fixed.

### RELATED APPLICATION INFORMATION

This application claims priority to U.S. Provisional Patent Application No. 63/009,690, filed on Apr. 14, 2020, incorporated herein by reference in its entirety.

### BACKGROUND

### Technical Field

The present invention relates to machine learning based medical systems and more particularly to a peptide-based vaccine generation system employing Generative Adversarial Networks (GANs) and drug property predictors.

### Description of the Related Art

Peptide-Major Histocompatibility Complex (MHC) protein interactions are essential in cell-mediated immunity, regulation of immune responses, and transplant rejection. Effective computational methods for peptide-MHC binding prediction will significantly reduce cost and time in clinical peptide vaccine search and design. Effective computational methods for peptide-protein binding prediction can greatly help clinical peptide vaccine search and design. Existing computational methods for peptide-MHC binding prediction can be roughly classified into two categories: linear regression-based methods and neural network (NN)-based methods. Almost all the previous computational systems focus on predicting a binding interaction score between a MHC protein and a given peptide but are incapable of generating strongly binding peptides given existing positive binding peptide examples.

### SUMMARY

According to aspects of the present invention, a computer-implemented method is provided for peptide-based vaccine generation. The method includes receiving a dataset of positive and negative binding peptide sequences. The method further includes pre-training a set of peptide binding property predictors on the dataset to generate training data. The method also includes training a Wasserstein Generative Adversarial Network (WGAN) only on the positive binding peptide sequences, in which a discriminator of the WGAN is updated to distinguish generated peptide sequences from sampled positive peptide sequences from the training data, and a generator of the WGAN is updated to fool the discriminator. The method additionally includes training the WGAN only on the positive binding peptide sequences while simultaneously updating the generator to minimize a kernel Maximum Mean Discrepancy (MMD) loss between the generated peptide sequences and the sampled peptide sequences and maximize prediction accuracies of a set of pre-trained peptide binding property predictors with parameters of the set of pre-trained peptide binding property predictors being fixed.

According to other aspects of the present invention, a computer program product is provided for peptide-based vaccine generation. The computer program product includes a non-transitory computer readable storage medium having program instructions embodied therewith. The program instructions are executable by a computer to cause the computer to perform a method. The method includes receiving a dataset of positive and negative binding peptide sequences. The method further includes pre-training a set of peptide binding property predictors on the dataset to generate training data. The method also includes training a Wasserstein Generative Adversarial Network (WGAN) only on the positive binding peptide sequences, in which a discriminator of the WGAN is updated to distinguish generated peptide sequences from sampled positive peptide sequences from the training data, and a generator of the WGAN is updated to fool the discriminator. The method additionally includes training the WGAN only on the positive binding peptide sequences while simultaneously updating the generator to minimize a kernel Maximum Mean Discrepancy (MMD) loss between the generated peptide sequences and the sampled peptide sequences and maximize prediction accuracies of a set of pre-trained peptide binding property predictors with parameters of the set of pre-trained peptide binding property predictors being fixed.

According to yet other aspects of the present invention, a computer processing system is provided for peptide-based vaccine generation. The system includes a memory device for storing program code. The system further includes a processor device operatively coupled to the memory device for running program code to receive a dataset of positive and negative binding peptide sequences. The processor device further runs the program code to pre-train a set of peptide binding property predictors on the dataset to generate training data. The processor device also runs the program code to train a Wasserstein Generative Adversarial Network (WGAN) only on the positive binding peptide sequences, in which a discriminator of the WGAN is updated to distinguish generated peptide sequences from sampled positive peptide sequences from the training data, and a generator of the WGAN is updated to fool the discriminator. The processor device additionally runs the program code to train the WGAN only on the positive binding peptide sequences while simultaneously updating the generator to minimize a kernel Maximum Mean Discrepancy (MMD) loss between the generated peptide sequences and the sampled peptide sequences and maximize prediction accuracies of a set of pre-trained peptide binding property predictors with parameters of the set of pre-trained peptide binding property predictors being fixed.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram showing an exemplary computing device, in accordance with an embodiment of the present invention;

FIGS. 2-3 are flow diagrams showing an exemplary training method for peptide-based vaccine generation, in accordance with an embodiment of the present invention;

FIG. 4 is a flow diagram showing an exemplary inference method for peptide-based vaccine generation, in accordance with an embodiment of the present invention;

FIG. 5 is a block diagram showing an exemplary discriminator, in accordance with an embodiment of the present invention;

FIG. 6 is a block diagram showing an exemplary property predictor, in accordance with an embodiment of the present invention;

FIG. 7 is a block diagram showing an exemplary generator, in accordance with an embodiment of the present invention; and

FIG. 8 is a block diagram showing an artificial neural network (ANN) architecture, in accordance with an embodiment of the present invention.

### DETAILED DESCRIPTION OF PREFERRED EMBODIMENTS

Embodiments of the present invention are directed to a peptide-based vaccine generation system employing Generative Adversarial Networks (GANs) and drug property predictors.

In one or more embodiments, a deep learning system is proposed for generating novel strong binding peptides to MHC proteins based on a dataset that includes both positive binding peptides and negative binding peptides. Instead of predicting binding scores of a pre-defined set of peptides as done traditionally, the present invention employs a trained Generative Adversarial Network (GAN) on positive binding peptides and one or many binding property predictors to generate new binding peptides interacting with MHC molecules.

Given a dataset that includes both positive and negative binding peptide sequences interacting with MHC, a Wasserstein Generative Adversarial Network is trained only on the positive binding peptide sequences. The Wasserstein GAN includes a generator and a discriminator. The generator is a deep neural network, which transforms a sampled latent code vector z from a standard multivariate unit-variance Gaussian distribution to a peptide feature representation matrix with each column corresponding to an amino acid. The discriminator is a deep neural network with local connections between the input representation layer and the first hidden layer and outputs a scalar as in a standard Wasserstein GAN. The term “deep neural network” refers to a neural network with several fully-connected layers. The parameters of the discriminator are updated to distinguish generated peptide sequences from sampled positive peptide sequences from the training data. The parameters of the generator are updated to fool the discriminator.

Besides optimizing the objective function of a Wasserstein GAN for generating positive binding peptide sequences, the present invention simultaneously updates the generator by minimizing a kernel Maximum Mean Discrepancy (MMD) loss between generated peptide sequences and sampled peptide sequences and maximizing the prediction accuracies of one or many pre-trained peptide binding property predictors. These peptide sequence predictors are pre-trained deep neural networks using the given positive and negative binding peptide sequences with corresponding supervision signals. These predictors can also be deep neural networks pre-trained on other user-specified peptide sequence datasets.

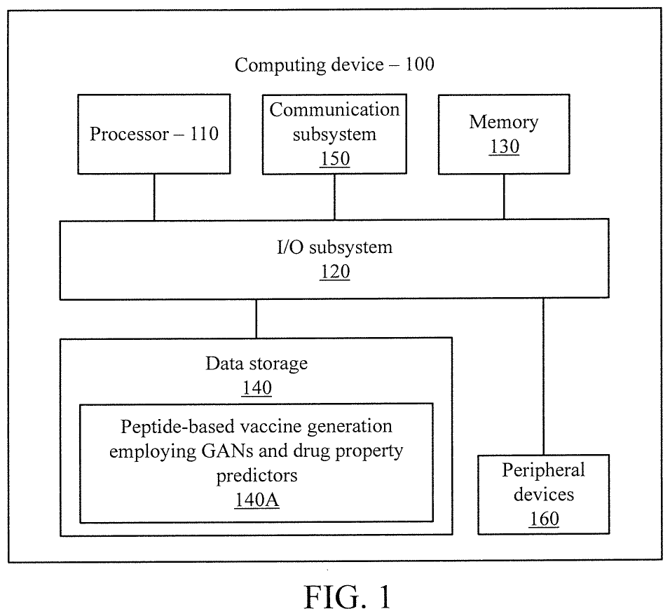

FIG. 1 is a block diagram showing an exemplary computing device 100, in accordance with an embodiment of the present invention. The computing device 100 is configured to perform peptide-based vaccine generation employing Generative Adversarial Networks (GANs) and drug property predictors.

The computing device 100 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 100 may be embodied as a one or more compute sleds, memory sleds, or other racks, sleds, computing chassis, or other components of a physically disaggregated computing device. As shown in FIG. 1, the computing device 100 illustratively includes the processor 110, an input/output subsystem 120, a memory 130, a data storage device 140, and a communication subsystem 150, and/or other components and devices commonly found in a server or similar computing device. Of course, the computing device 100 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 130, or portions thereof, may be incorporated in the processor 110 in some embodiments.

The processor 110 may be embodied as any type of processor capable of performing the functions described herein. The processor 110 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 130 may be embodied as any type of volatile or non-volatile memory or data storage capable of performing the functions described herein. In operation, the memory 130 may store various data and software used during operation of the computing device 100, such as operating systems, applications, programs, libraries, and drivers. The memory 130 is communicatively coupled to the processor 110 via the I/O subsystem 120, which may be embodied as circuitry and/or components to facilitate input/output operations with the processor 110 the memory 130, and other components of the computing device 100. For example, the I/O subsystem 120 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.) and/or other components and subsystems to facilitate the input/output operations. In some embodiments, the I/O subsystem 120 may form a portion of a system-on-a-chip (SOC) and be incorporated, along with the processor 110, the memory 130, and other components of the computing device 100, on a single integrated circuit chip.

The data storage device 140 may be embodied as any type of device or devices configured for short-term or long-term storage of data such as, for example, memory devices and circuits, memory cards, hard disk drives, solid state drives, or other data storage devices. The data storage device 140 can store program code for peptide-based vaccine generation employing Generative Adversarial Networks (GANs) and drug property predictors. The communication subsystem 150 of the computing device 100 may be embodied as any network interface controller or other communication circuit, device, or collection thereof, capable of enabling communications between the computing device 100 and other remote devices over a network. The communication subsystem 150 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 100 may also include one or more peripheral devices 160. The peripheral devices 160 may include any number of additional input/output devices, interface devices, and/or other peripheral devices. For example, in some embodiments, the peripheral devices 160 may include a display, touch screen, graphics circuitry, keyboard, mouse, speaker system, microphone, network interface, and/or other input/output devices, interface devices, and/or peripheral devices.

Of course, the computing device 100 may also include other elements (not shown), as readily contemplated by one of skill in the art, as well as omit certain elements. For example, various other input devices and/or output devices can be included in computing device 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 100 are readily contemplated by one of ordinary skill in the art given the teachings of the present invention provided herein.

As employed herein, the term “hardware processor subsystem” or “hardware processor” can refer to a processor, memory (including RAM, cache(s), and so forth), software (including memory management software) or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), FPGAs, and/or PLAs.

These and other variations of a hardware processor subsystem are also contemplated in accordance with embodiments of the present invention

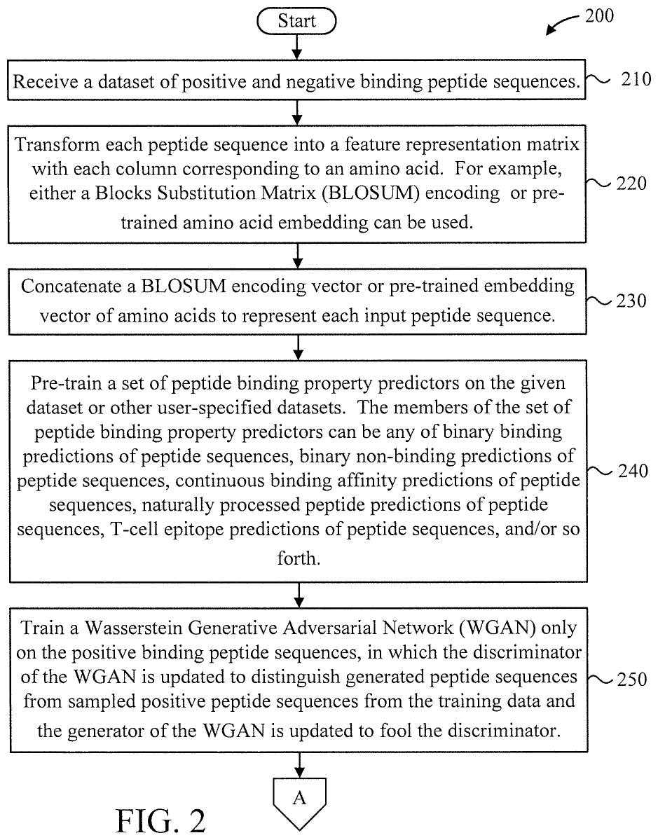

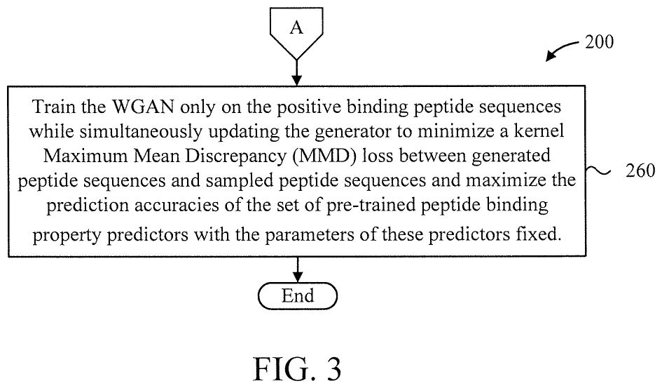

FIGS. 2-3 are flow diagrams showing an exemplary training method 200 for peptide-based vaccine generation, in accordance with an embodiment of the present invention.

At block 210, receive a dataset of positive and negative binding peptide sequences.

At step 220, transform each peptide sequence into a feature representation matrix with each column corresponding to an amino acid. For example, in an embodiment, either a Blocks Substitution Matrix (BLOSUM) encoding or pre-trained amino acid embedding can be used.

At block 230, concatenate a BLOSUM encoding vector or pre-trained embedding vector of amino acids to represent each input peptide sequence.

At block 240, pre-train a set of peptide binding property predictors on the given dataset or other user-specified datasets. The members of the set of peptide binding property predictors can be any of binary binding predictions of peptide sequences, binary non-binding predictions of peptide sequences, continuous binding affinity predictions of peptide sequences, naturally processed peptide predictions of peptide sequences, T-cell epitope predictions of peptide sequences, and/or so forth.

At block 250, train a Wasserstein Generative Adversarial Network (WGAN) only on the positive binding peptide sequences, in which the discriminator of the WGAN is updated to distinguish generated peptide sequences from sampled positive peptide sequences from the training data and the generator of the WGAN is updated to fool the discriminator.

At block 260, train the WGAN only on the positive binding peptide sequences while simultaneously updating the generator to minimize a kernel Maximum Mean Discrepancy (MMD) loss between generated peptide sequences and sampled peptide sequences and maximize the prediction accuracies of the set of pre-trained peptide binding property predictors with the parameters of these predictors fixed.

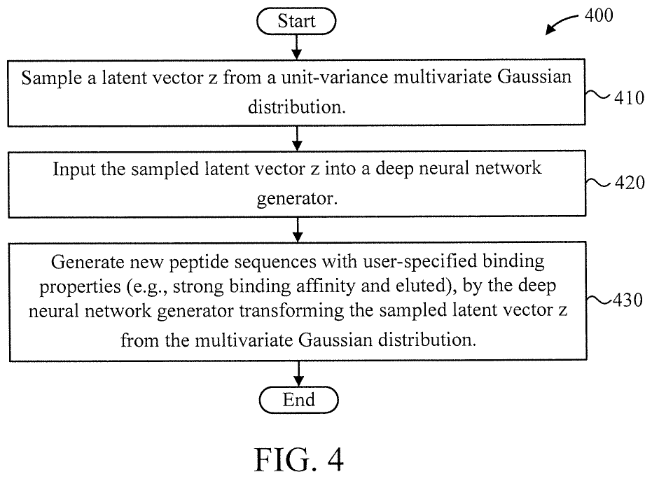

FIG. 4 is a flow diagram showing an exemplary inference method 400 for peptide-based vaccine generation, in accordance with an embodiment of the present invention.

At block 410, sample a latent vector z from a unit-variance multivariate Gaussian distribution.

At block 420, input the sampled latent vector z into a deep neural network generator.

At block 430, generate new peptide sequences with user-specified binding properties (e.g., strong binding affinity and eluted), by the deep neural network generator transforming the sampled latent vector z from the multivariate Gaussian distribution.

A vaccine can be administered to a patient based on the results of block 430.

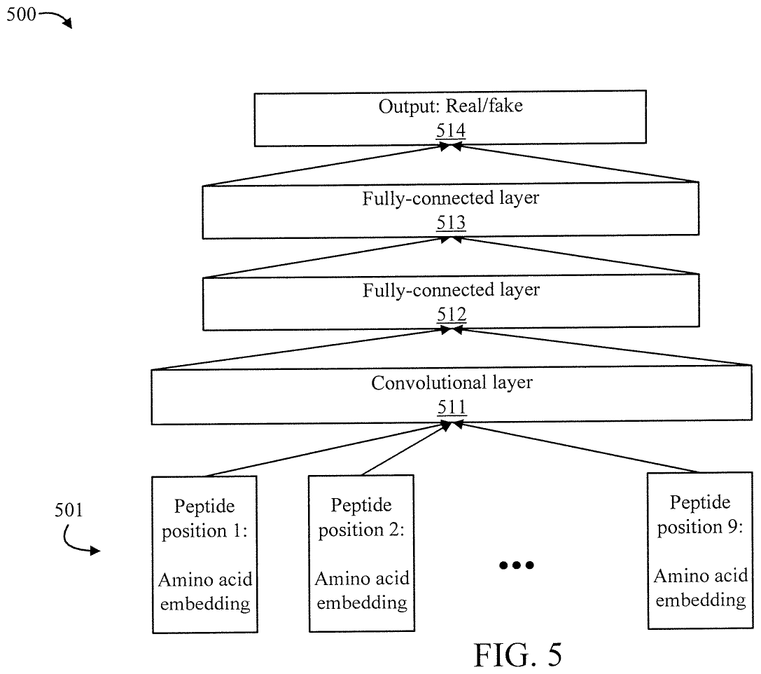

FIG. 5 is a block diagram showing an exemplary discriminator 500, in accordance with an embodiment of the present invention.

The discriminator 500 receives an input peptide sequence matrix with amino acid embeddings 501, and includes a convolutional layer 511, a fully connected layer 512, a fully connected layer 513, and an output layer 514 outputting real/fake sequences. The input peptide sequence matrix is a d-by-n matrix, in which n is the length of the input peptide (for example, n=9 for most MHC Class I positive binding peptides), d is a user-specified dimensionality of amino acid embedding vectors, and the i-th column of the matrix corresponds to the embedding vector of the i-th amino acid in the input peptide sequence.

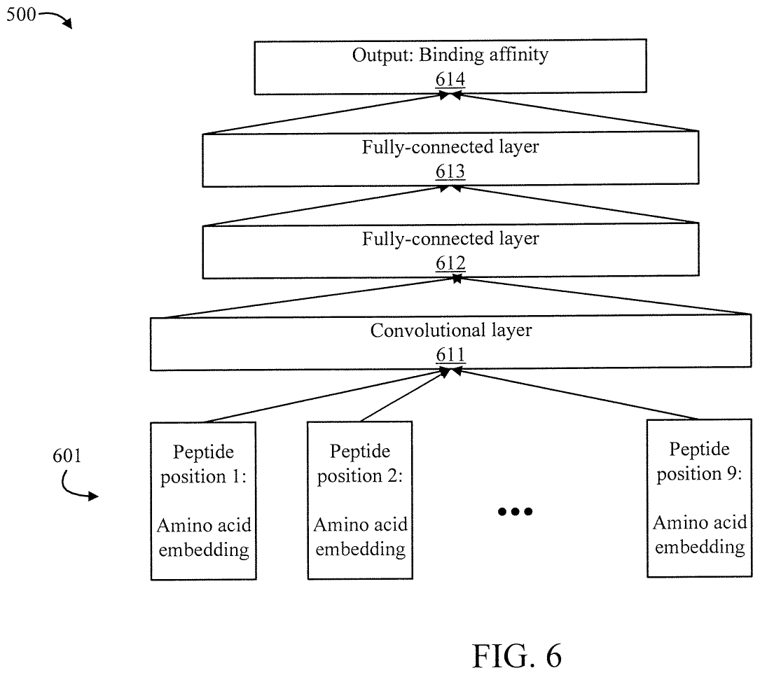

FIG. 6 is a block diagram showing an exemplary property predictor 600, in accordance with an embodiment of the present invention.

The property predictor 600 receives an input peptide sequence matrix with amino acid embeddings 601, and includes a convolutional layer 611, a fully connected layer 612, a fully connected layer 613, and an output layer 614 outputting a binding affinity. The input peptide sequence matrix is a d-by-n matrix, in which n is the length of the input peptide (for example, n=9 for most MHC Class I binding peptides), d is a user-specified dimensionality of amino acid embedding vectors, and the i-th column of the matrix correspond to the embedding vector of the i-th amino acid in the input peptide sequence.

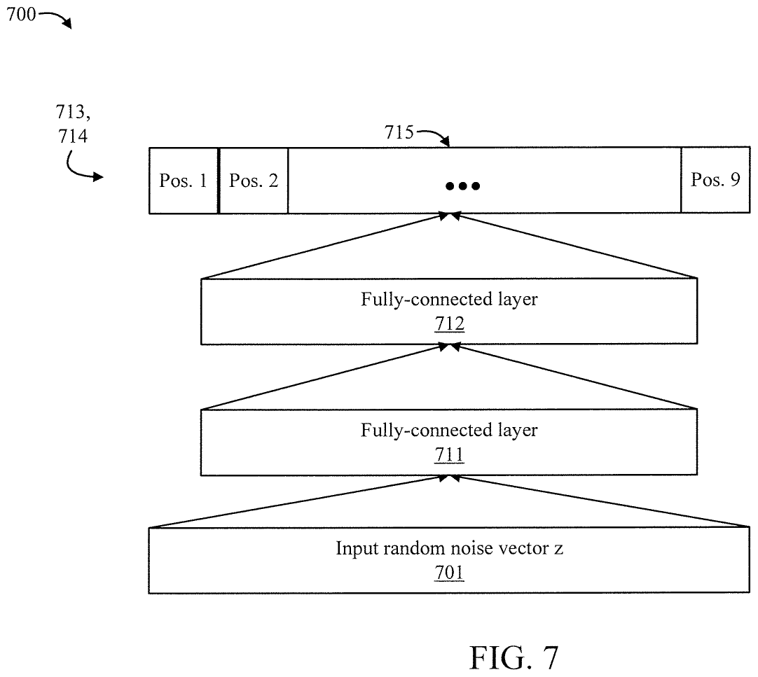

FIG. 7 is a block diagram showing an exemplary generator 700, in accordance with an embodiment of the present invention.

The generator 700 receives an input random noise vector z 701, and includes a fully connected layer 711, a fully connected layer 712, and an output layer 713 outputting softmax output units 714. The softmax output units 714 are concatenated into a Peptide sequence 715. Specifically, to generate a peptide sequence with length n, we have n output softmax units with each unit corresponding to a position in the peptide sequence. Each softmax unit outputs 20 probabilities summing to 1, which denotes the emitting probabilities of 20 amino acids. Ideally, in a softmax unit i corresponding to position i of a positive binding peptide sequence, the emitting probability of the ground-truth amino acid should be close to 1, and all the other 19 emitting probabilities of this softmax unit should be close to 0.

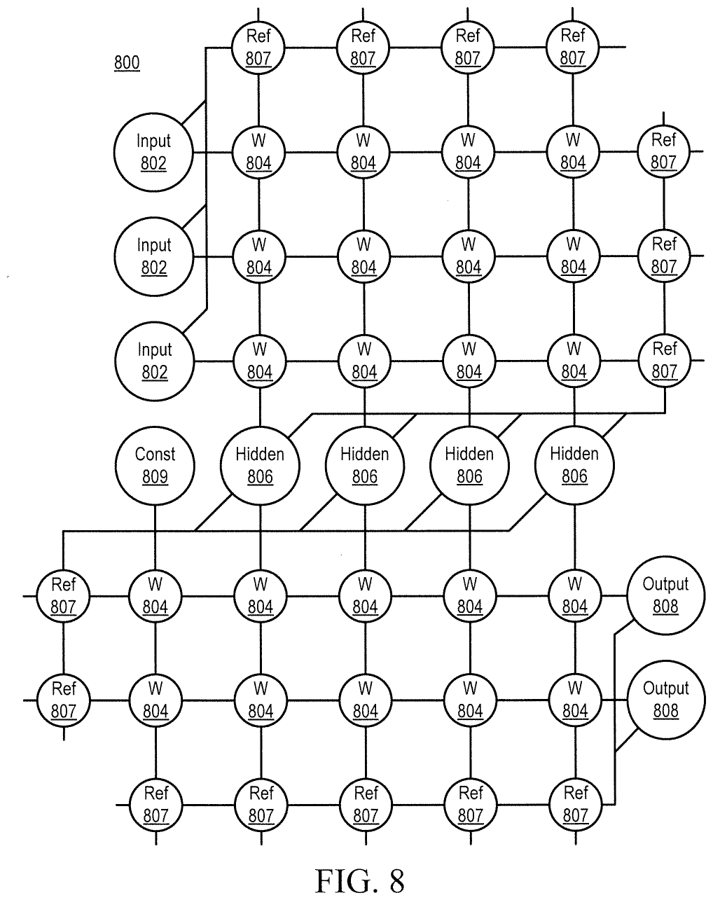

FIG. 8 is a block diagram showing an artificial neural network (ANN) architecture 800, in accordance with an embodiment of the present invention. It should be understood that the present architecture is purely exemplary and that other architectures or types of neural network may be used instead. The ANN embodiment described herein is included with the intent of illustrating general principles of neural network computation at a high level of generality and should not be construed as limiting in any way.

Furthermore, the layers of neurons described below and the weights connecting them are described in a general manner and can be replaced by any type of neural network layers with any appropriate degree or type of interconnectivity. For example, layers can include convolutional layers, pooling layers, fully connected layers, softmax layers, or any other appropriate type of neural network layer. Furthermore, layers can be added or removed as needed and the weights can be omitted for more complicated forms of interconnection.

During feed-forward operation, a set of input neurons 802 each provide an input signal in parallel to a respective row of weights 804. The weights 804 each have a respective settable value, such that a weight output passes from the weight 804 to a respective hidden neuron 806 to represent the weighted input to the hidden neuron 806. In software embodiments, the weights 804 may simply be represented as coefficient values that are multiplied against the relevant signals. The signals from each weight adds column-wise and flows to a hidden neuron 806.

The hidden neurons 806 use the signals from the array of weights 804 to perform some calculation. The hidden neurons 806 then output a signal of their own to another array of weights 804. This array performs in the same way, with a column of weights 804 receiving a signal from their respective hidden neuron 806 to produce a weighted signal output that adds row-wise and is provided to the output neuron 808.

It should be understood that any number of these stages may be implemented, by interposing additional layers of arrays and hidden neurons 806. It should also be noted that some neurons may be constant neurons 809, which provide a constant output to the array. The constant neurons 809 can be present among the input neurons 802 and/or hidden neurons 806 and are only used during feed-forward operation.

During back propagation, the output neurons 808 provide a signal back across the array of weights 804. The output layer compares the generated network response to training data and computes an error. The error signal can be made proportional to the error value. In this example, a row of weights 804 receives a signal from a respective output neuron 808 in parallel and produces an output which adds column-wise to provide an input to hidden neurons 806. The hidden neurons 806 combine the weighted feedback signal with a derivative of its feed-forward calculation and stores an error value before outputting a feedback signal to its respective column of weights 804. This back propagation travels through the entire network 800 until all hidden neurons 806 and the input neurons 802 have stored an error value.

During weight updates, the stored error values are used to update the settable values of the weights 804. In this manner the weights 804 can be trained to adapt the neural network 800 to errors in its processing. It should be noted that the three modes of operation, feed forward, back propagation, and weight update, do not overlap with one another.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for peptide-based vaccine generation, comprising:
receiving a dataset of positive and negative binding peptide sequences;
pre-training a set of peptide binding property predictors on the dataset to generate training data;
training a Wasserstein Generative Adversarial Network (WGAN) only on the positive binding peptide sequences, in which a discriminator of the WGAN is updated to distinguish generated peptide sequences from sampled positive peptide sequences from the training data, and a generator of the WGAN is updated to fool the discriminator; and
training the WGAN only on the positive binding peptide sequences while simultaneously updating the generator to minimize a kernel Maximum Mean Discrepancy (MMD) loss between the generated peptide sequences and the sampled peptide sequences and maximize prediction accuracies of a set of pre-trained peptide binding property predictors with parameters of the set of pre-trained peptide binding property predictors being fixed.

2. The computer-implemented method of claim 1, further comprising concatenating a vector of amino acids to represent each of the positive and negative binding peptides.

3. The computer-implemented method of claim 2, wherein the concatenated vector is a Blocks Substitution Matrix (BLOSUM) encoding vector of amino acids.

4. The computer-implemented method of claim 2, wherein the concatenated vector is a pre-trained embedding vector of amino acids.

5. The computer-implemented method of claim 1, wherein the set of peptide binding property predictors is selected pre-trained on the dataset or other user-specified datasets.

6. The computer-implemented method of claim 1, wherein members of the set of peptide binding property predictors are selected from a group consisting of binary binding predictions of peptide sequences, binary non-binding predictions of peptide sequences, continuous binding affinity predictions of peptide sequences, naturally processed peptide predictions of peptide sequences, and T-cell epitope predictions of peptide sequences.

7. The computer-implemented method of claim 1, wherein the discriminator is implemented by a first deep neural network having a convolutional layer and a fully-connected layer, and the generator is implemented by a second deep neural network having a fully-connected layer.

8. The computer-implemented method of claim 1, further comprising generating peptide-based vaccines with user-specified properties using the trained WGAN.

9. The computer-implemented method of claim 1, wherein the peptide-based vaccines are output from the generator as softmax output units, and wherein the generator comprises a fully-connected layer for receiving an input random noise vector and outputting the softmax output units.

10. A computer program product for peptide-based vaccine generation, the computer program product comprising a non-transitory computer readable storage medium having program instructions embodied therewith, the program instructions executable by a computer to cause the computer to perform a method comprising:
receiving a dataset of positive and negative binding peptide sequences;
pre-training a set of peptide binding property predictors on the dataset to generate training data;
training a Wasserstein Generative Adversarial Network (WGAN) only on the positive binding peptide sequences, in which a discriminator of the WGAN is updated to distinguish generated peptide sequences from sampled positive peptide sequences from the training data, and a generator of the WGAN is updated to fool the discriminator; and
training the WGAN only on the positive binding peptide sequences while simultaneously updating the generator to minimize a kernel Maximum Mean Discrepancy (MMD) loss between the generated peptide sequences and the sampled peptide sequences and maximize prediction accuracies of a set of pre-trained peptide binding property predictors with parameters of the set of pre-trained peptide binding property predictors being fixed.

11. The computer program product of claim 10, wherein the method further comprises concatenating a vector of amino acids to represent each of the positive and negative binding peptides.

12. The computer program product of claim 11, wherein the concatenated vector is a Blocks Substitution Matrix (BLOSUM) encoding vector of amino acids.

13. The computer program product of claim 11, wherein the concatenated vector is a pre-trained embedding vector of amino acids.

14. The computer program product of claim 10, wherein the set of peptide binding property predictors is selected pre-trained on the dataset or other user-specified datasets.

15. The computer program product of claim 10, wherein members of the set of peptide binding property predictors are selected from a group consisting of binary binding predictions of peptide sequences, binary non-binding predictions of peptide sequences, continuous binding affinity predictions of peptide sequences, naturally processed peptide predictions of peptide sequences, and T-cell epitope predictions of peptide sequences.

16. The computer program product of claim 10, wherein the discriminator is implemented by a first deep neural network having a convolutional layer and a fully-connected layer, and the generator is implemented by a second deep neural network having a fully-connected layer.

17. The computer program product of claim 10, wherein the method further comprises generating peptide-based vaccines with user-specified properties using the trained WGAN.

18. The computer program product of claim 10, wherein the peptide-based vaccines are output from the generator as softmax output units, and wherein the generator comprises a fully-connected layer for receiving an input random noise vector and outputting the softmax output units.

19. A computer processing system for peptide-based vaccine generation, comprising:
a memory device for storing program code;
a processor device operatively coupled to the memory device for running program code to:
receive a dataset of positive and negative binding peptide sequences;
pre-train a set of peptide binding property predictors on the dataset to generate training data;
train a Wasserstein Generative Adversarial Network (WGAN) only on the positive binding peptide sequences, in which a discriminator of the WGAN is updated to distinguish generated peptide sequences from sampled positive peptide sequences from the training data, and a generator of the WGAN is updated to fool the discriminator; and
train the WGAN only on the positive binding peptide sequences while simultaneously updating the generator to minimize a kernel Maximum Mean Discrepancy (MMD) loss between the generated peptide sequences and the sampled peptide sequences and maximize prediction accuracies of a set of pre-trained peptide binding property predictors with parameters of the set of pre-trained peptide binding property predictors being fixed.

20. The computer-implemented method of claim 19, further comprising generating peptide-based vaccines with user-specified properties using the trained WGAN.
